(19) United States
(12) Patent Application Publication
(10) Pub. No.: US 2011/0201953 A1
(43) Pub. Date: Aug. 18, 2011

(54) METHOD AND SYSTEM FOR PATIENT EVALUATION

(75) Inventors: Willi Kaiser, Emmendingen (DE); Martin Findeis, Freiburg (DE); Rolf Band, Gottenheim (DE)

(73) Assignee: GENERAL ELECTRIC COMPANY, Schenectady, NY (US)

(21) Appl. No.: 12/706,563

(22) Filed: Feb. 16, 2010

(51) Int. Cl. A61B 5/0402

(52) U.S. Cl. 600/514

(57) ABSTRACT

A method includes receiving audio files from a computer using a portable electronic device. The audio files are electrocardiograms obtained during exercise tests performed on patients and converted to an audio format. The method also includes performing audio playback of the audio files, receiving user input at the portable electronic device regarding an analysis of the patients based on the playback of the audio files, and compiling a list of a group of the patients selected for further investigation based on the user input.

### FIELD OF THE INVENTION

This disclosure relates generally to a method and system for evaluating a patient.

### BACKGROUND OF THE INVENTION

An electrocardiograph is a cardiac diagnostic/monitoring system adapted to record the electrical activity of a patient's heart. The electrocardiograph generally includes an array of sensors or transducers placed at predetermined positions on a patient's body. An electrocardiograph is commonly implemented during exercise tests wherein a patient is evaluated while undergoing some form of strenuous physical activity such as, for example, running on a treadmill or pedaling a stationary exercise bicycle.

The recorded data from an electrocardiograph is generally displayed in the form of a graph that is often referred to as an electrocardiogram (ECG). It is well known that the visual analysis of the various waves that make up an ECG can yield important diagnostic information. For example, an ECG signal obtained from a patient during an exercise test may be analyzed by visually evaluating the constituent PQRST complex segments. A visual analysis, however, may not reveal all the information contained in the ECG from the exercise test. Furthermore, the visual analysis may not be the most efficient means for obtaining certain types of ECG information, such as longer term ECG information.

### BRIEF DESCRIPTION OF THE INVENTION

The above-mentioned shortcomings, disadvantages and problems are addressed herein which will be understood by reading and understanding the following specification.

In an embodiment, a method includes receiving audio files from a computer using a portable electronic device. The audio files are electrocardiograms obtained during exercise tests performed on patients and converted to an audio format. The method also includes performing audio playback of the audio files, receiving user input at the portable electronic device regarding an analysis of the patients based on the playback of the audio files, and compiling a list of a group of the patients selected for further investigation based on the user input.

In another embodiment, a method includes obtaining an electrocardiogram during an exercise test performed on a patient, converting the electrocardiogram to an audio file, transferring the audio file to a portable electronic device for playback, performing audio playback of the audio file, and receiving user input at the portable electronic device regarding an analysis of the patient based on the audio playback.

In another embodiment, a system includes a computer configured to obtain an electrocardiogram from a patient undergoing an exercise test, and to convert the electrocardiogram into an audio file. The system also includes a portable electronic device configured to receive the audio file from the computer, to perform audio playback of the audio file, and to receive user input regarding an analysis of the patient based on the playback of the audio file.

Various other features, objects, and advantages of the invention will be made apparent to those skilled in the art from the accompanying drawings and detailed description thereof.

### BRIEF DESCRIPTION OF THE DRAWINGS

FIG. 1 is a schematic illustration of a cardiac exercise system in accordance with an embodiment;

FIG. 2 is an illustration of a graphical user interface in accordance with an embodiment; and

FIG. 3 is a flow chart illustrating a method in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

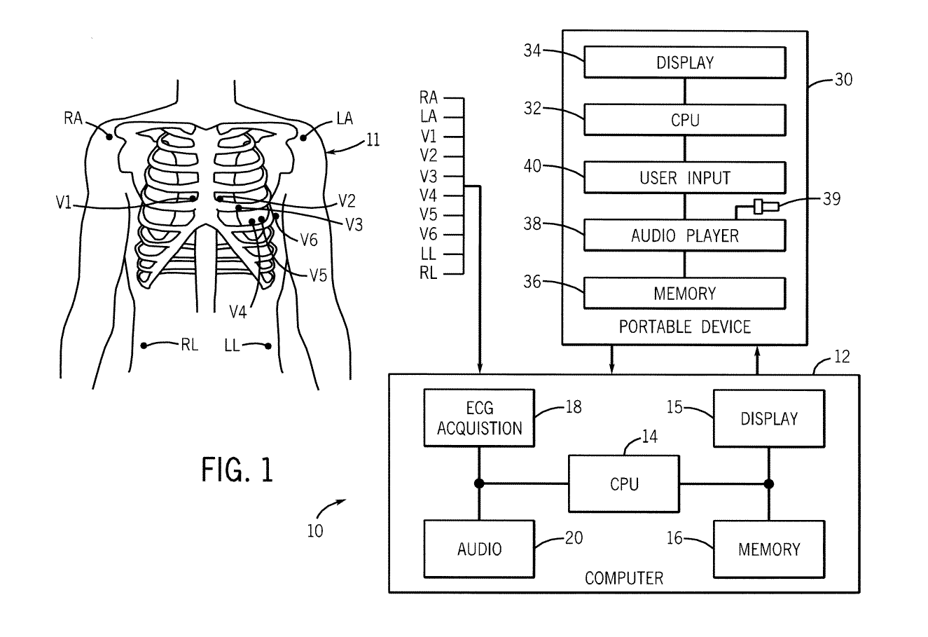

Referring to FIG. 1, a schematically represented cardiac exercise system 10 is shown. Cardiac exercise system 10 is generally configured to record the electrical activity of a patient's heart in the form of an ECG. By way of example, the ECG may be recorded during an exercise test. The exercise test may include an exercise phase, wherein a patient 11 is undergoing some form of strenuous physical activity such as, for example, running on a treadmill, and may also include a recovery phase wherein the patient 11 is relaxing. ECG data obtained during an exercise test includes a high density of information that is potentially relevant to patient risk prediction and early disease detection.

Cardiac exercise system 10 may, for example, include a computer 12 having a central processing unit (CPU) 14 coupled to a display 15 and a storage or memory device 16. Computer 12 also includes an ECG acquisition component 18. ECG acquisition component 18 may include, for example, signal acquisition hardware (e.g., signal amplifiers, galvanic isolation components, analog-to-digital converters, etc.) and a software application executed by CPU 14 to receive digital ECG data from the signal acquisition hardware and calculate ECG leads. ECG acquisition component 18 is configured to measure an electrical signal generated by a patient's heart. To facilitate this measurement, Cardiac exercise system 10 can be coupled to the patient 11 by an array of sensors or transducers. In the illustrated embodiment, the array of sensors include a right arm electrode RA; a left arm electrode LA; chest electrodes V1, V2, V3, V4, V5 and V6; a right leg electrode RL; and a left electrode leg LL for acquiring a standard twelve lead, ten-electrode ECG signal. The twelve ECG leads include leads I, II, V1, V2, V3, V4, V5 and V6 which are acquired directly from the patient leads, and leads III, aVR, aVL and aVF which are derived using Einthoven's law. In other embodiments, alternative configurations of sensors and sensor locations can be used to acquire a standard or non-standard ECG signal. For example, an expanded fifteen lead system, including four extra electrodes, can be used to form Frank X, Y and Z leads.

Computer 12 is also configured to transfer ECG data to audio component 20 for conversion to an audio file as will be described in further detail below. Computer 12 is also configured to facilitate the selection of ECG data files for conversion to audio files as well as storage and management of the converted audio files. For example, computer 12 may interface with ECG acquisition component 18 to provide a graphical user interface (e.g., by using the various operating system functions of a WINDOWS XP operating system residing on computer 12) that allows a user to assign an identifier, such as a patient ID, to each ECG, and to select particular ECGs for conversion to audio files that may be transferred to a portable electronic device as will be described below. Computer 12 is further configured to provide ECG data to display 15 in a format that can be visually analyzed, and to facilitate selection and management of multiple ECGs during visual analysis.

The computer 12 may also contain an audio component 20. Audio component 20 is configured to convert ECG data to an audio file, such as, for example, a “.wav file” or a “.mp3 file” that can be stored in memory device 16 and/or transferred to a portable electronic device. As will be described in detail hereinafter, the ECG can be converted into an audio file, and an auditory evaluation of the converted audio file can be performed for purposes such as patient risk prediction and disease detection.

ECG data obtained during an exercise test and converted to an audio file can be assessed in a relatively short amount of time (e.g., in several seconds). By way of example, the audio file may have a reproduction factor of either 60 or 120. For purposes of this disclosure, a “reproduction factor” refers to the differential between the audio file playback time and the time during which the recorded events actually took place. Therefore, a reproduction factor of 60 means that an audio file containing a 10-minute exercise test can be played back in 10 seconds. Advantageously, this allows a listener to evaluate more information in a shorter amount of time and thereby more quickly assess patient 11. According to another embodiment, the audio file can be played back in stereo such that the left channel represents the lead V2 (shown in FIG. 1) and the right channel represents the lead V5 (shown in FIG. 1). According to yet another embodiment, the audio file can be played back in stereo such that the left channel represents the lead aVF (shown in FIG. 1) and the right channel represents the lead V2.

Cardiac exercise system 10 may also include a portable electronic device 30. Portable electronic device 30 may be, for example, a handheld PDA or cellular telephone configured to execute software applications. Portable electronic device 30 may include a CPU 32 coupled to a display 34 and a storage or memory device 36. Portable electronic device 30 may also contain an audio player 38 operatively connected to a speaker 39 (e.g., a speaker mounted within the portable electronic device or an external speaker, such as a headphone) for playback of audio files. In particular, portable electronic device 30 is configured to provide audio playback of and manage ECG data that has been converted to an audio format by computer 12 and transferred to portable electronic device 30, using, for example, a Universal Serial Bus (USB) connection or a wireless upload and stored in memory device 36.

In order to facilitate the afore-mentioned playback and management of audio files comprising ECG data, Portable electronic device 30 may also include a user input component 40. User input component 40 may be a software application executed by CPU 32, such as, for example, a hypertext markup language (html) file executed on Microsoft Corporation's WINDOWS MOBILE operating system or an executable program developed for use with or Apple's iPHONE OS software. By way of example, user input component 40 may be configured to provide a graphical user interface for display on display 34 of portable electronic device 30.

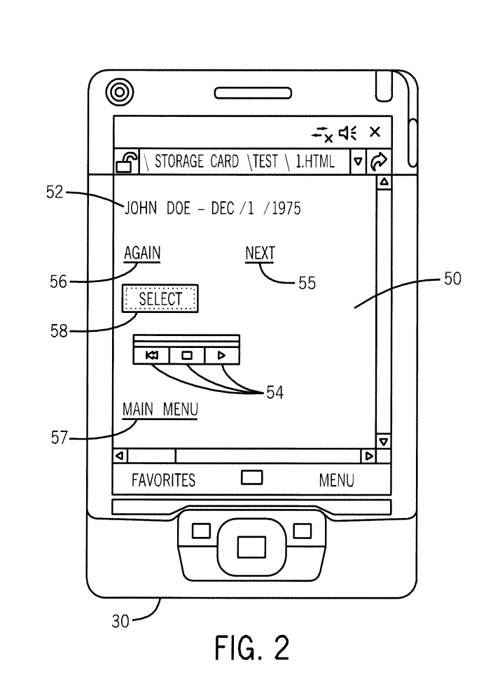

Referring to FIG. 2, a graphical user interface implemented by user input component 40 is shown. Upon activation of user input component 40, a screen 50 may be displayed on display 34 and playback of audio files stored in memory device 36 may be initiated via an interface with audio player 38. Screen 50 may include an identifier 52 including information such as patient ID and date to identify a particular audio file currently set for playback. Screen 50 may also include an input selection 55 configured to advance to the next audio file to be played. Screen 50 may also include an input selection 56 configured to repeat playback of the current audio file. Screen 50 may further include a main menu input selection 57 configured to display a list of patients for which audio files have been transferred to portable electronic device 30 and which are available for audio playback. Using input selection 57, a user can directly access an audio file for a particular patient. Screen 50 may also include audio playback controls 54 to allow, for example, stopping and resuming playback.

Screen 50 may also include an input selection 58 configured to facilitate designation of the audio file as being associated with a patient selected for further analysis. Such designation may be based on an auditory analysis of the playback, including for example, the various auditory analyses described in further detail below with respect to normal, borderline and abnormal auditory evaluations. Upon receiving input via input selection 58, user input component 40 may add the patient associated with the audio file to a list of patients selected for further analysis, such as a visual analysis of the ECG using computer 12. The patient list may be, for example, a text file stored in memory 36 for transfer to computer 12. The user may then, for example, use input selection 55 to advance to the next audio file to be played.

Referring again to FIG. 1, computer 12 may be further configured to receive the patient list from portable electronic device 30, using, for example, a Universal Serial Bus (USB) connection or a wireless upload, and to store the patient list in memory device 16. Computer 12 may be also be configured to display the patient list on display 15 in a visual format, such as a graphical user interface (e.g., using a WINDOWS operating system) that allows a user to select a particular patient from the patient list, and to display the ECG for that patient on display 15 for visual analysis.

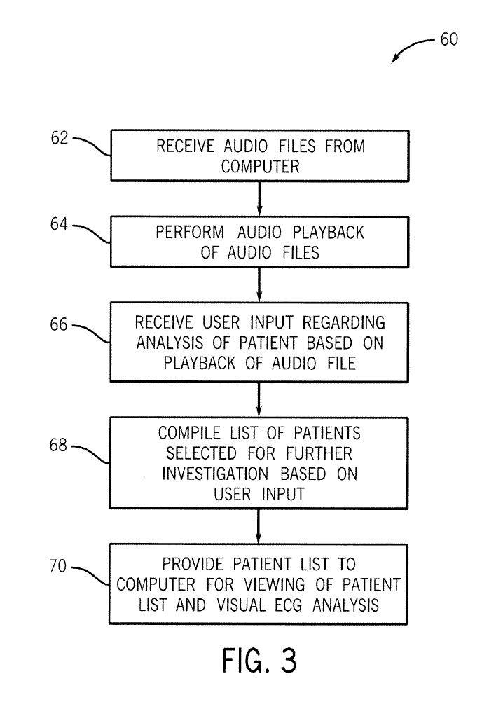

Referring to FIG. 3, a flow chart illustrates a method 60 in accordance with an embodiment. The individual blocks shown in FIG. 3 represent steps that may be performed in accordance with the method 60. Unless otherwise specified, steps 62-70 of the method 60 need not be performed in the order shown.

At a step 62, audio files are uploaded to portable electronic device 30 from computer 12 using, for example, a USB connection. Each of the audio files includes data obtained during an exercise test performed on a patient and converted from an ECG to an audio format using audio application 20. For example, the ECG may be obtained during an exercise phase and during an immediately subsequent recovery phase and may be obtained, for example, with a ten-electrode array. The ECG data may then be converted to an audio file, such as, for example, a “.wav file” or a “.mp3 file” that can be stored in the memory device 16 for future playback on portable electronic device 30 via audio player 38.

At a step 64, audio playback of one or more of the transferred audio files is performed using audio player 38 in order to facilitate an auditory analysis of the ECG data. For example, user input component 40 on portable electronic device 30 may be executed to initiate playback of one or more of the audio files stored in memory 36 via audio player 38, as well as to provide a graphical user interface to facilitate playback and selection via input selections 55, 56, and 58. As described above, the audio file may, for example, have a reproduction factor of either 60 or 120 and may be played back in stereo such that the left channel represents the lead V2 (shown in FIG. 1) and the right channel represents the lead V5 (shown in FIG. 1). According to yet another embodiment, the audio file may be played back in stereo such that the left channel represents the lead aVF (shown in FIG. 1) and the right channel represents the lead V2.

As described above, the analysis may include an auditory evaluation of the audio file performed by an experienced professional healthcare provider such as a physician proficient at identifying a wide variety of audible abnormalities that are relevant to patient risk prediction and/or disease detection. The analysis may include, for example, any of the various auditory analyses described above with respect to normal, borderline and abnormal auditory evaluations.

At a step 66, user input regarding the analysis of the patient based on the playback of the audio file is received at the portable electronic device 30. For example, user input component 40 on portable electronic device 30 may provide graphical user interface screen 50 to facilitate receiving the user input. After listening to playback of an audio file for a particular patient, the user may provide input such as an instruction via input selection 55 to advance to the next audio file to be played if the current audio file was not of interest, or an instruction to repeat playback of the current audio file via input selection 56 if the correct analysis is uncertain after the initial audio playback. The user may also provide an instruction via input selection 58 to select a patient for further investigation based on the auditory evaluation. For example, the user may wish to exclude any patients having a normal evaluation while selecting those patients having either a borderline or abnormal evaluation for further investigation (e.g., a visual analysis of the patient's ECG on computer 12) based on the audio playback of the ECG data. After selecting a particular patient for further investigation, the user may then provide an instruction via input selection 55 to advance to the next audio file to be played. The user input may also include an instruction via input selection 57 to display a list of all patients for which audio files have been transferred to portable electronic device 30 and which are available for audio playback so that the user can directly access an audio file for a particular patient.

At a step 68, a list of a group of the patients selected for further investigation based on the user input is compiled and stored in memory 36. The patient list may include, for example, a list of patients having either borderline or abnormal evaluations for further investigation.

At a step 70, the patient list is provided to computer 12 so that the list may be viewed and particular patient ECGs may be selected and visually analyzed. For example, the user may upload the patient list from portable electronic device 30, to display the patient list on display 15, select a particular patient from the patient list, and display the patient's ECG on display 15 for visual analysis.

Using the above-described system or method, a user such as a physician may more efficiently access and analyze ECG data. In particular, the user may listen to and analyze the audio ECG data over a broader range of times and locations by leveraging the schedule flexibility and portability enabled by the use of a portable electronic device such as a PDA or cellular telephone. The user may also perform a greater number of analyses due to the shortened time period for analysis enabled by the use of an audio file. The user can also save time by identifying and marking patients of interest and performing a visual analysis only in those instances where an audio analysis indicates, for example, a borderline or abnormal evaluation. The user may also reduce the risk that important arrhythmia events such as atrial fibrillations will be overlooked in a visual analysis

The following section will provide non-limiting illustrative examples of “normal” auditory evaluations, “abnormal” auditory evaluations, and “borderline” auditory evaluations. For purposes of this disclosure, a “normal” auditory evaluation is one wherein the evaluation does not indicate an increased patient risk or the presence of a disease. An “abnormal” auditory evaluation is one wherein the evaluation strongly indicates an increased patient risk or the presence of a disease, and a “borderline” auditory evaluation is one wherein there is a somewhat weaker indication of increased patient risk or the presence of a disease. In the examples described hereinafter, the patient 11 (shown in FIG. 1) undergoes an exercise test using, for example, a treadmill or exercise bicycle, and is subjected to a series of events that increase the patient's requisite activity level in a generally stepwise manner. These events may, for example, include an increase in treadmill speed and/or incline angle, or an increase in bicycle load.

According to an illustrative embodiment, a normal auditory evaluation is one wherein the patient's ECG includes the following audibly detectable characteristics. A first audibly detectable characteristic of a normal evaluation is a generally stepwise increase in heart rate wherein each heart rate increase corresponds to an increase in requisite activity level induced by the exercise test. As previously indicated, the increase in requisite activity level may, for example, be induced by increasing the speed or incline angle of a treadmill. Another audibly detectable characteristic of a normal evaluation is a heart rate that is generally proportional to activity level. In other words, the patient's heart rate steadily increases during the exercise phase and the patient's heart rate steadily decreases during the recovery phase of the exercise test. An increase or decrease in the patient's heart rate is audibly detectable as a corresponding increase or decrease in the frequency of the sound wave representing the patient's ECG.

According to an illustrative embodiment, an abnormal auditory evaluation is one wherein the patient's ECG includes the following audibly detectable characteristics. A first audibly detectable characteristic of an abnormal evaluation is a generally linear increase of heart rate in response to a generally stepwise increase in activity level. This characteristic is abnormal in that the patient's heart rate does not directly respond to an increase in activity level. Another audibly detectable characteristic of an abnormal evaluation is a heart rate that does not steadily decreases during the recovery phase of the exercise test. Some additional audibly detectable conditions indicative of an abnormal auditory evaluation will hereinafter be individually described.

A ventricular premature beat (VPB) or extrasystole is an audibly detectable condition consistent with an abnormal auditory evaluation. As is known to those skilled in the art, a VPB is a form of irregular heartbeat in which the ventricle contracts prematurely. During an exercise test, the beat-to-beat intervals should steadily decrease during the exercise phase and steadily increase during the recovery phase. A VPB interrupts this behavior with a short beat-to-beat interval followed by a long beat-to-beat interval (a compensatory pause). This irregularity is clearly audible. A cumulative appearance of VPBs in the recovery phase is an indicator for an increased risk of mortality.

T-wave alternans (TWA) refers to a condition wherein there are alternating variations in shape of consecutive T-waves. During TWA, the ECG generally comprises a plurality of even numbered T-waves having a first generally common shape, and a plurality of odd numbered T-waves having a second generally common shape wherein the first shape is distinct from the second shape. The alternating variations in the shape of consecutive T-waves, which are indicative of TWA, are audibly detectable as an additional deeper tone.

Atrial fibrillation (AF) is an abnormal heart rhythm that is associated with stroke and cardiovascular events, and for which there is increase prevalence in elderly patients. Heartbeats in a normal heart begin after electricity generated in the atria by the sinoatrial node spreads through the heart and causes contraction of the heart muscle and pumping of blood. In AF, the regular electrical impulses of the sinoatrial node are replaced by disorganized, rapid electrical impulses that result in irregular heart beats. AF is audibly detectable as a blurring noise caused by inconsistent or irregular durations between consecutive heart beats.

According to an illustrative embodiment, a borderline auditory evaluation is one wherein the patient's ECG includes VPBs during the exercise phase of the exercise test.

While the invention has been described with reference to preferred embodiments, those skilled in the art will appreciate that certain substitutions, alterations and omissions may be made to the embodiments without departing from the spirit of the invention. Accordingly, the foregoing description is meant to be exemplary only, and should not limit the scope of the invention as set forth in the following claims.

I claim:

1. A method comprising:
receiving audio files from a computer using a portable electronic device, wherein the audio files are electrocardiograms obtained during exercise tests performed on patients and converted to an audio format;
performing audio playback of the audio files;
receiving user input at the portable electronic device regarding an analysis of the patients based on the playback of the audio files; and
compiling a list of a group of the patients selected for further investigation based on the user input.

2. The method of claim 1, wherein each of the electrocardiograms is a twelve lead, ten-electrode electrocardiogram.

3. The method of claim 1, further comprising providing a graphical user interface on the portable audio device to facilitate performing the audio playback and receiving the user input.

4. The method of claim 1, wherein receiving the user input includes receiving an instruction to perform a repeat of the audio playback for at least one of the audio files.

5. The method of claim 1, further comprising providing a list of the audio files received from the computer by the portable electronic device.

6. The method of claim 1, wherein playing the audio file includes playing the audio file in stereo.

7. The method of claim 6, wherein playing the audio file in stereo includes playing the audio file such that a first stereo channel represents a first electrocardiograph lead and a second stereo channel represents a second electrocardiograph lead.

8. A method comprising:
obtaining an electrocardiogram during an exercise test performed on a patient;
converting the electrocardiogram to an audio file;
transferring the audio file to a portable electronic device for playback;
performing audio playback of the audio file; and
receiving user input at the portable electronic device regarding an analysis of the patient based on the audio playback.

9. The method of claim 8, wherein obtaining the electrocardiogram includes obtaining a twelve lead, ten-electrode electrocardiogram.

10. The method of claim 8, further comprising providing a graphical user interface on the portable audio device to facilitate performing the audio playback and receiving the user input.

11. The method of claim 8, wherein receiving the user input includes receiving an instruction to perform a repeat of the audio playback.

12. The method of claim 8, wherein the audio file is a first electrocardiogram audio file, and wherein receiving the user input includes receiving an instruction to perform audio playback of a second electrocardiogram audio file.

13. The method of claim 8, wherein receiving the user input includes receiving an instruction to select the patient for further investigation.

14. The method of claim 13, further comprising compiling a patient list, wherein the patient list includes the patient selected for further investigation.

15. A system comprising:
a computer configured to obtain an electrocardiogram from a patient undergoing an exercise test, and to convert the electrocardiogram into an audio file; and
a portable electronic device configured to receive the audio file from the computer, to perform audio playback of the audio file, and to receive user input regarding an analysis of the patient based on the playback of the audio file.

16. The system of claim 15, wherein the electrocardiogram is a twelve lead, ten-electrode electrocardiogram.

17. The system of claim 15, wherein the portable electronic device includes a graphical user interface to facilitate performing the audio playback and receiving the user input.

18. The system of claim 15, wherein the user input is an instruction to perform a repeat of the audio playback.

19. The system of claim 15, wherein the user input is an instruction to select the patient for further investigation.

20. The system of claim 19, wherein the portable electronic device is further configured to compile a patient list, wherein the patient list includes the patient selected for further investigation.
